(19) United States
(12) Patent Application Publication
(10) Pub. No.: US 2017/0020421 A1
(43) Pub. Date: Jan. 26, 2017

(54) METHOD, APPARATUS AND NON-TRANSITORY COMPUTER-READABLE RECORDING MEDIUM FOR MEASURING PHOTOPLETHYSMOGRAPHY SIGNALS

(71) Applicant: HUINNO CO., LTD., Seoul (KR)

(72) Inventors: Min-Yong Shin, Suwon (KR); Yoon Chul Choi, Seongnam (KR); Heung Jong Yoo, Seongnam (KR); Seong Jun Shin, Seongnam (KR)

(73) Assignee: HUINNO CO., LTD.

(21) Appl. No.: 15/215,178

(22) Filed: Jul. 20, 2016

(30) Foreign Application Priority Data
Jul. 20, 2015 (KR) 10-2015-0102694
Feb. 26, 2016 (KR) 10-2016-0023600

(51) Int. Cl. A61B 5/1455; A61B 5/00

(52) U.S. Cl. CPC A61B 5/14552; A61B 5/7203; A61B 5/7221; A61B 5/7278; A61B 2562/0238

(57) ABSTRACT

There are provided a method, apparatus and non-transitory computer-readable recording medium for measuring photoplethysmography (PPG) signals. Accurate PPG signals can be obtained even when brightness of ambient light is not constant due to external light sources, and accuracy of a variety of biological information derivable from the PPG signals can be improved.

### CROSS-REFERENCE TO RELATED APPLICATIONS

This application claims priority to Korean Application No. 10-2015-0102694, filed Jul. 20, 2015, and Korean Application No. 10-2016-0023600, filed Feb.26, 2016. The applications are incorporated herein by reference.

### FIELD OF THE INVENTION

The present invention relates to methods, apparatuses, and non-transitory computer-readable recording media for measuring photoplethysmography signals.

### BACKGROUND

Due to recent rapid progress in science and technology, the quality of life of all mankind is being enhanced and medical environment has changed a great deal. In the past, once a medical image was taken by means such as X-ray, CT, fMRI or the like, it would take several hours or days to be able to interpret the image.

However, a picture archive communication system (PACS) has been introduced to enable a medical image to be taken and then transmitted to a monitor screen of a radiology specialist for prompt interpretation. Further, medical equipment for ubiquitous healthcare are wide spread so that patient-performed self-checks on blood glucose and blood pressure are feasible at anytime and anywhere out of hospital, and diabetic or hypertensive patients use the equipment at their home and/or office. Particularly in the case of hypertension, which is one of the principal causes of various diseases and whose prevalence rate is increasing, there is a need for a system for consistent measurement and real-time notification of blood pressure, and various types of studies associated therewith are being attempted.

Meanwhile, biological information such as an electrocardiogram, heart rate, body temperature, blood oxygen saturation level, electromyogram, sweat gland activity, sweat rate, and respiratory rate is obtained on the basis of biosignals respectively acquired from two or more contacts on a human body (not necessarily adjoining each other physically), and thus a technique for properly processing and measuring the biosignals acquired from the contacts on the body is required to obtain biological information.

Photoplethysmography (PPG) signals are significantly utilized in measuring a variety of biological information on cardiac functions, including blood oxygen saturation levels (SpO₂). According to conventional PPG signal measurement techniques that have been used so far, there are technical constraints such as a need for a shielding structure for preventing errors caused by external light sources.

As a technique for measuring a blood oxygen saturation level (SpO₂) using PPG signals, it is possible to sense visible light (e.g., red light, green light, etc.) and infrared light reflected from a human body and calculate an oxygen saturation level on the basis of PPG signals, each corresponding to the sensed visible light and infrared light.

The technique is based on a principle that light absorptivity of oxyhemoglobin (HBO₂) in blood is greater for infrared light than for visible light.

FIG. 1 illustrates a situation in which an oxygen saturation level is measured according to the prior art.

Referring to FIG. 1, a light receiver 110 of a conventional PPG signal measurement apparatus receives not only light irradiated onto a body part 120 of a user by a light emitter (not shown) and reflected from the body part of the user, but also ambient light irradiated from an external light source 130 such as the sun or a lamp. Because the intensity or brightness of the ambient light irradiated from the external light source 130 can vary greatly according to measurement conditions, it can be difficult to maintain the amount (intensity or brightness) of light received by the light receiver 110.

The brightness (i.e., illuminance) of light sensed by the light receiver 110 needs to be constantly maintained in order to accurately measure PPG signals (and an oxygen saturation level). In order to address this problem, a shielding structure has been employed in the prior art to shield the parts where light is irradiated and sensed from the external light source. According to the prior art, this causes spatial constraints requiring the shield structure to contain all of the components including the light emitter for irradiating light and the light receiver for sensing light, which causes the size of the measurement apparatus to become excessively large due to the shielding structure.

Therefore, a technique for accurately measuring PPG signals (and an oxygen saturation level) in a situation in which brightness of ambient light is not constant due to external light sources is desirable.

### SUMMARY

Provided herein are methods, apparatuses, and non-transitory computer-readable recording media for accurately measuring photoplethysmography (PPG) signals in a situation in which brightness of ambient light is not constant due to external light sources, by irradiating light of a first wavelength range and light of a second wavelength range onto a body part of a user through a first filter and a second filter, respectively; sensing light of the first wavelength range entering through the first filter and light of the second wavelength range entering through the second filter, respectively; measuring a first illuminance of the light of the first wavelength range entering through the first filter and a second illuminance of the light of the second wavelength range entering through the second filter, respectively; generating a first PPG signal corresponding to the sensed light of the first wavelength range and a second PPG signal corresponding to the sensed light of the second wavelength range; and adjusting brightness of at least one of the light of the first wavelength range and the light of the second wavelength range irradiated onto the body part of the user, such that a difference between a predetermined reference illuminance and at least one of the first and second measured illuminances is less than a predetermined level.

According to one exemplary embodiment, there is provided a method for measuring photoplethysmography (PPG) signals, including the steps of: irradiating light of a first wavelength range and light of a second wavelength range onto a body part of a user, respectively; sensing light of the first wavelength range entering through a first filter and light of the second wavelength range entering through a second filter, respectively, and measuring a first illuminance of the light of the first wavelength range entering through the first filter and a second illuminance of the light of the second wavelength range entering through the second filter, respectively; and generating a first PPG signal corresponding to the sensed light of the first wavelength range and a second PPG signal corresponding to the sensed light of the second wavelength range, wherein, in the irradiating step, brightness of at least one of the light of the first wavelength range and the light of the second wavelength range irradiated onto the body part of the user is adjusted such that a difference between a predetermined reference illuminance and at least one of the first and second measured illuminances is less than a predetermined level.

According to another exemplary embodiment, there is provided an apparatus for measuring photoplethysmography (PPG) signals, including: a first light emitter and a second light emitter configured to irradiate light of a first wavelength range and light of a second wavelength range onto a body part of a user, respectively; a first light receiver and a second light receiver configured to sense light of the first wavelength range entering through a first filter and light of the second wavelength range entering through a second filter, respectively; a first illuminance sensor and a second illuminance sensor configured to measure a first illuminance of the light of the first wavelength range entering through the first filter and a second illuminance of the light of the second wavelength range entering through the second filter, respectively; a calculator configured to generate a first PPG signal corresponding to the sensed light of the first wavelength range and a second PPG signal corresponding to the sensed light of the second wavelength range; and a controller configured to adjust brightness of at least one of the light of the first wavelength range and the light of the second wavelength range irradiated onto the body part of the user, such that a difference between a predetermined reference illuminance and at least one of the first and second measured illuminances is less than a predetermined level.

In addition, there are further provided other embodiments including methods and apparatuses, as well as non-transitory computer-readable recording media having stored thereon computer programs for executing the methods.

PPG signals may be accurately measured even in a situation in which brightness of ambient light is not constant due to external light sources. Accuracy of a variety of biological information derivable from PPG signals may be improved.

Brightness of light irradiated onto a human body may be adaptively adjusted, thereby preventing spatial constraints caused by a conventional shielding structure and allowing a PPG signal measurement apparatus to be easily installed in a wearable device having a small size and limited shape.

### BRIEF DESCRIPTION OF THE DRAWINGS

FIG. 1 illustrates a situation in which photoplethysmography (PPG) signals are measured according to the prior art.

FIG. 2 schematically shows the configuration of an entire system according to one embodiment herein. FIG. 3 illustrates the internal configuration of a PPG signal measurement apparatus according to one embodiment.

FIG. 4 illustrates the appearance of the PPG signal measurement apparatus according to one embodiment.

FIG. 5 illustrates how PPG signals and an oxygen saturation level are measured according to one embodiment.

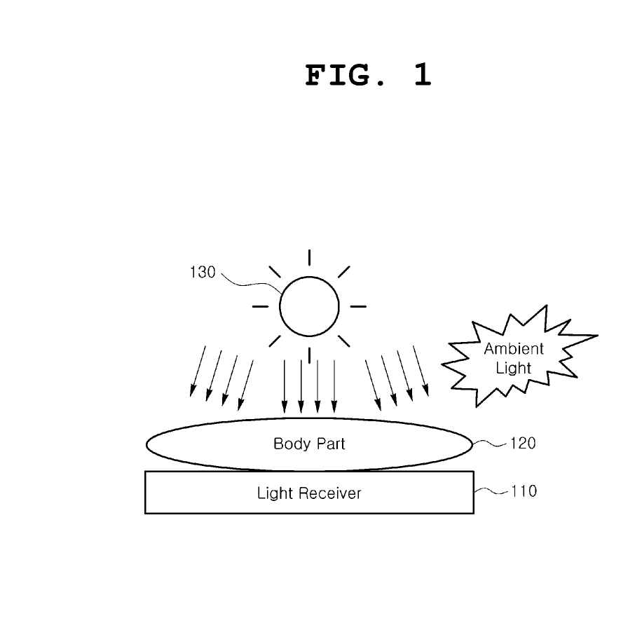

### DETAILED DESCRIPTION

In the following detailed description of the present invention, references are made to the accompanying drawings that show, by way of illustration, specific embodiments in which the invention may be practiced. These embodiments are described in sufficient detail to enable those skilled in the art to practice the invention. It is to be understood that the various embodiments of the invention, although different from each other, are not necessarily mutually exclusive. For example, specific shapes, structures and characteristics described herein may be implemented as modified from one embodiment to another without departing from the spirit and scope of the invention. Furthermore, it shall be understood that the locations and/or arrangements of individual elements within each of the disclosed embodiments may also be modified without departing from the spirit and scope of the invention. Therefore, the following detailed description is not to be taken in a limiting sense, and the scope of the invention, if properly described, is limited only by the appended claims together with all equivalents thereof. In the drawings, like reference numerals refer to the same or similar functions throughout the several views.

Although one or more exemplary embodiments are described as using a plurality of units to perform the exemplary processes, it is understood that the exemplary processes can also be performed by one or plurality of modules. Additionally, it is understood that the term controller/control unit refers to a hardware device that includes a memory and a processor. The memory is configured to store the modules and the processor is specifically configured to execute said modules to perform one or more processes which are described further below. Any units described herein can be devices and/or structures that are configured to perform the stated functions.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

An entire system for measuring photoplethysmography (PPG) signals according to one embodiment will be discussed in detail below.

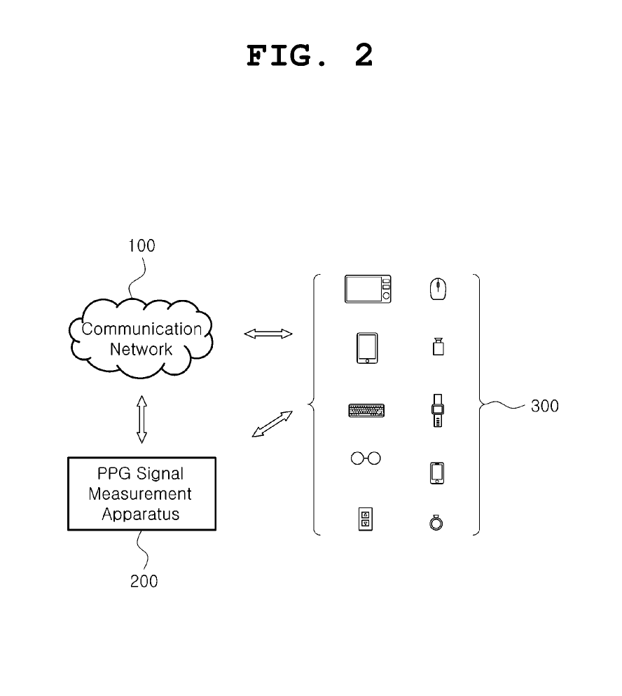

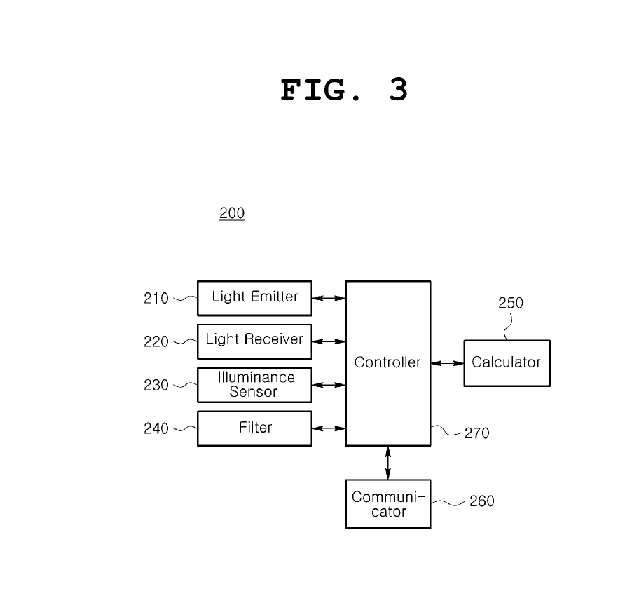

FIG. 2 schematically shows the configuration of the entire system according to one embodiment.

As shown in FIG. 2, the entire system according to one embodiment can include a communication network 100, a PPG signal measurement apparatus 200, and a device 300.

First, the communication network 100 according to one embodiment can be implemented regardless of communication modality such as wired and wireless communications, and can be constructed from a variety of communication networks, such as local area networks (LANs), metropolitan area networks (MANs), and wide area networks (WANs). The communication network 100 described herein can include known short-range wireless communication networks such as Wi-Fi, Wi-Fi Direct, LTE Direct, and Bluetooth. However, the communication network 100 is not necessarily limited thereto, and may at least partially include known wired/wireless data communication networks, known telephone networks, or known wired/wireless television communication networks.

Next, the PPG signal measurement apparatus 200 according to one embodiment can function to accurately measure PPG signals and an oxygen saturation level in a situation in which brightness of ambient light is not constant due to external light sources, by irradiating light of a first wavelength range and light of a second wavelength range onto a body part of a user through a first filter 241 and a second filter 242, respectively; sensing light of the first wavelength range entering through the first filter 241 and light of the second wavelength range entering through the second filter 242, respectively; measuring a first illuminance of the light of the first wavelength range entering through the first filter 241 and a second illuminance of the light of the second wavelength range entering through the second filter 242, respectively; generating a first PPG signal corresponding to the sensed light of the first wavelength range and a second PPG signal corresponding to the sensed light of the second wavelength range; and adjusting brightness of at least one of the light of the first wavelength range and the light of the second wavelength range irradiated onto the body part of the user, so that a difference between a predetermined reference illuminance and at least one of the first and second measured illuminances is less than a predetermined level.

The functions of the PPG signal measurement apparatus 200 will be discussed in more detail below. Meanwhile, although the PPG signal measurement apparatus 200 has been described as above, the above description is illustrative and it will be apparent to those skilled in the art that at least some of the functions or components required for the PPG signal measurement apparatus 200 can be implemented or included in the device 300, as necessary.

Lastly, the device 300 according to one embodiment is digital equipment that can function to connect to and then communicate with the PPG signal measurement apparatus 200, and any type of digital equipment having a memory means and a microprocessor for computing capabilities can be adopted as the device 300. The device 300 can be a wearable device such as a smart glass, a smart watch, a smart band, a smart ring, and/or a smart necklace, or can be a device such as a smart phone, a smart pad, a desktop computer, a notebook computer, a workstation, a personal digital assistant (PDA), a web pad, and/or a mobile phone. According to one embodiment, the device 300 can include a sensing means, such as one or more sensors, for acquiring a biosignal from a human body, and a display means, such as one or more displays, for providing biological information to a user. In addition, according to one embodiment, the device 300 can further include an application program for performing the functions provided herein. The application can reside in the device 300 in the form of a program module. The nature of the program module can be generally similar to those of a calculator 250, a communicator 260, and a controller 270 of the PPG signal measurement apparatus 200 to be described below. Here, at least a part of the application can be replaced with a hardware and/or firmware device that can perform substantially equal or equivalent functions, as necessary.

Configuration of the PPG Signal Measurement Apparatus

Hereinafter, the internal configuration of the PPG signal measurement apparatus 200 configured in part to implement some or all of the functions of the respective components thereof will be discussed.

FIG. 3 illustrates the internal configuration of the PPG signal measurement apparatus according to one embodiment.

Referring to FIG. 3, the PPG signal measurement apparatus 200 according to one embodiment can include a light emitter 210, a light receiver 220, an illuminance sensor 230, a filter 240, a calculator 250, a communicator 260, and a controller 270. According to one embodiment, at least some of the calculator 250, the communicator 260, and the controller 270 can be program modules to communicate with an external system (not shown). The program modules can be included in the PPG signal measurement apparatus 200 in the form of operating systems, application program modules and other program modules, while they may be physically stored in a variety of known storage devices. Further, the program modules can also be stored in a remote storage device that can communicate with the PPG signal measurement apparatus 200. Meanwhile, such program modules can include, but are not limited to, routines, subroutines, programs, objects, components, data structures and the like for performing specific tasks or executing specific abstract data types as will be described below.

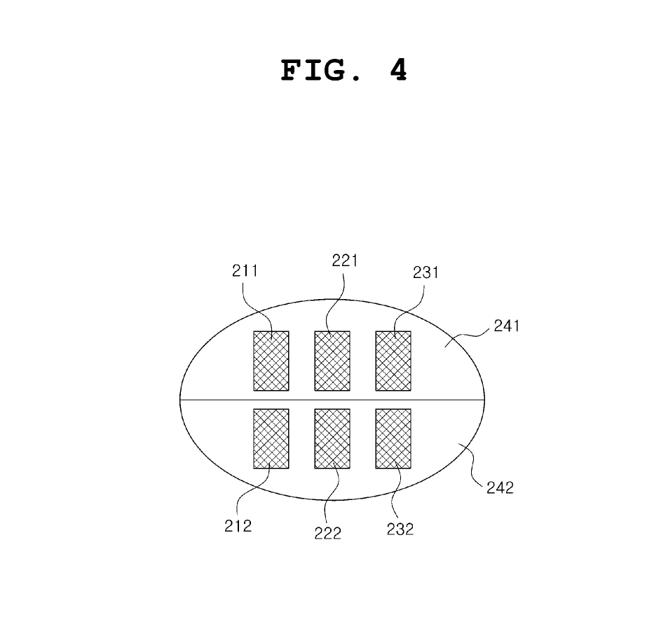

FIG. 4 illustrates the appearance of the PPG signal measurement apparatus according to one embodiment.

First, according to one embodiment, the light emitter 210 can function to irradiate light of a first wavelength range and light of a second wavelength range onto a body part (e.g., a finger, wrist, etc.) of a user for which measurement is to be carried out. Specifically, the light emitter 210 according to one embodiment can include a first light emitter 211 for emitting light of the first wavelength range and a second light emitter 212 for emitting light of the second wavelength range, and can consist of light emitting diodes (LEDs) for generating the light of the first wavelength range or the light of the second wavelength range according to a predetermined cycle. For example, the light of the first wavelength range can include visible light of a wavelength range of about 490 nm to 780 nm, and the light of the second wavelength range can include infrared light of a wavelength range of about 800 nm to 980 nm.

Further, according to one embodiment, the light of the first wavelength range and the light of the second wavelength range emitted from the light emitter 210 may be irradiated onto the body part of the user through the first filter 241 and the second filter 242, respectively. Here, the first and second filters 241 and 242 may consist of filters for selectively transmitting the light of the first wavelength range and the light of the second wavelength range, respectively.

Furthermore, according to one embodiment, brightness of the light of the first wavelength range or the light of the second wavelength range irradiated onto the body part of the user can be adjusted such that a first or a second illuminance measured by the illuminance sensor 230 matches a predetermined reference illuminance, as will be described below. That is, according to one embodiment, the brightness of at least one of the light of the first wavelength range and the light of the second wavelength range irradiated onto the body part of the user can be adaptively adjusted so that the difference between at least one of the first and second illuminances and the predetermined reference illuminance is less than a predetermined level.

For example, when the first illuminance measured by a first illuminance sensor 231 is less than the predetermined reference illuminance, the brightness of the light of the first wavelength range irradiated by the first light emitter 211 may be increased such that the first illuminance matches the predetermined reference illuminance. For another example, when the second illuminance measured by a second illuminance sensor 232 is greater than the predetermined reference illuminance, the brightness of the light of the second wavelength range irradiated by the second light emitter 212 may be reduced such that the second illuminance matches the predetermined reference illuminance. According to one embodiment, the function of adjusting the brightness of the light of the first wavelength range or the light of the second wavelength range may be performed by the controller 270.

Next, according to one embodiment of the invention, the light receiver 220 can function to sense light of the first wavelength range and light of the second wavelength range, respectively. Specifically, the light receiver 220 according to one embodiment of the invention can include a first light receiver 221 for sensing light of the first wavelength range and a second light receiver 222 for sensing light of the second wavelength range, and can include photodiodes for sensing the light of the first wavelength range or the light of the second wavelength range. According to one embodiment, the light sensed by the light receiver 220 can include the light irradiated by the light emitter 210 and reflected from the body part of the user and also ambient light irradiated from external light sources.

Further, according to one embodiment, the light of the first wavelength range and the light of the second wavelength range sensed by the light receiver 220 can enter through the first and second filters 241 and 242, respectively. As described above, the first and second filters 241 and 242 can include filters for selectively transmitting the light of the first wavelength range and the light of the second wavelength range, respectively.

Next, according to one embodiment, the illuminance sensor 230 can function to measure a first illuminance of the light of the first wavelength range entering through the first filter 241 and a second illuminance of the light of the second wavelength range entering through the second filter 242, respectively. Specifically, the illuminance sensor 230 according to one embodiment of the invention can include a first illuminance sensor 231 for sensing the first illuminance and a second illuminance sensor 232 for sensing the second illuminance, and the first and second illuminance sensors 231 and 232 can be disposed around the first and second light receivers 221 and 222, respectively.

Next, according to one embodiment, the calculator 250 may function to generate a first PPG signal corresponding to the light of the first wavelength range and a second PPG signal corresponding to the light of the second wavelength range.

More specifically, as the brightness of the light of the first wavelength range generated by the first light emitter 211 or the light of the second wavelength range generated by the second light emitter 212 is adaptively adjusted by the controller 270, the calculator 250 according to one embodiment can generate a first PPG signal corresponding to the light of the first wavelength range sensed by the first light receiver 221 and/or a second PPG signal corresponding to the light of the second wavelength range sensed by the second light receiver 222, when the first and/or second illuminance matches a predetermined reference illuminance such that the difference between the first and/or second illuminance and the predetermined reference illuminance is less than a predetermined level. Further, when the first or second illuminance exceeds the predetermined reference illuminance, the calculator 250 according to one embodiment can correct (or scale) the intensity of the first and/or second PPG signal with reference to the relative ratio of the first and/or second illuminance and the predetermined reference illuminance.

For example, when the first illuminance measured by the first illuminance sensor 231 is about 2,000 lux and the predetermined reference illuminance is about 1,000 lux, then the intensity of the first PPG signal corresponding to the light of the first wavelength range sensed by the first light receiver 221 can be scaled by about ½.

PPG signals can thus be accurately measured in a situation in which brightness of ambient light is not constant due to external light sources, without employing a conventional shielding structure causing spatial constraints and consequently making accurate measurement easier, simpler, and less expensive.

Meanwhile, according to one embodiment, the calculator 250 may function to calculate a blood oxygen saturation level in the body part of the user with reference to the first and second PPG signals generated as above.

Specifically, the calculator 250 according to one embodiment can calculate the oxygen saturation level on the basis of a general model for oxygen saturation level calculation, which is applicable when the illuminance of the sensed light matches the predetermined reference illuminance. The first and second PPG signals, which are obtained by adaptively controlling the brightness of the light generated by the light emitter 210 or by adaptively correcting the PPG signals generated from the light sensed by the light receiver 220 as described above, can be applied right to the general model for oxygen saturation level calculation, and thus the calculator 250 according to one embodiment can calculate the oxygen saturation level with reference to the first and second PPG signals corrected as above and the general model for oxygen saturation level calculation.

For example, the model for oxygen saturation level calculation according to one embodiment can be a model for calculating oxygen content of hemoglobin in blood on the basis of the difference between AC components of the first PPG signal (i.e., the signal corresponding to red light) and the second PPG signal (i.e., the signal corresponding to infrared light) corrected as above. However, it is noted that the model for oxygen saturation level calculation is not limited thereto, and can be changed without limitation as long as the objects discussed above can be achieved.

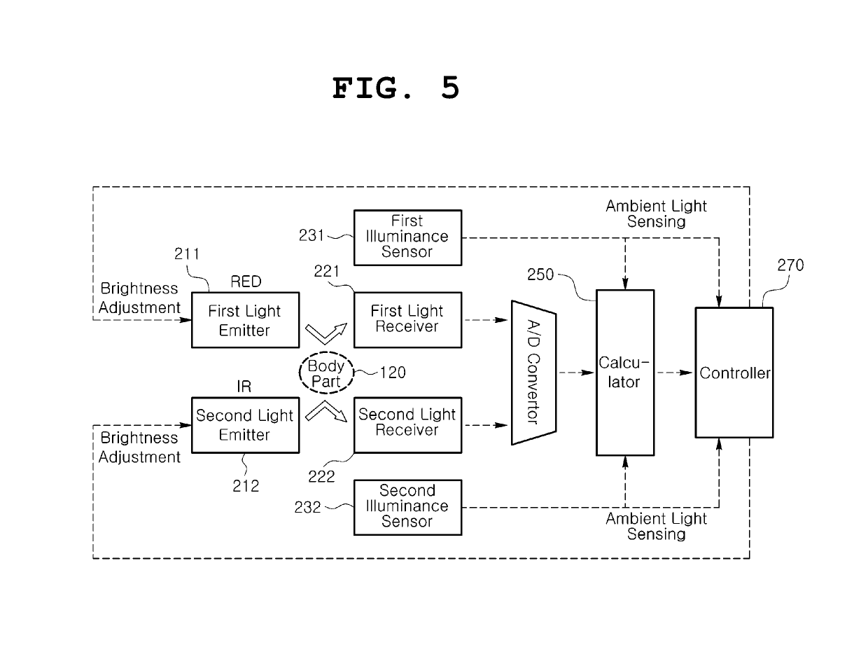

FIG. 5 illustrates how PPG signals and an oxygen saturation level are measured according to one embodiment.

Referring to FIG. 5, the first and second light emitters 211 and 212 according to one embodiment herein can generate and irradiate red light of a first wavelength range and infrared (IR) light of a second wavelength range onto a body part 120 of a user, respectively. The first and second light receivers 221 and 222 can sense red light of the first wavelength range and infrared light of the second wavelength range reflected from the body part of the user or irradiated from external light sources, respectively.

Referring further to FIG. 5, the first and second illuminance sensors 231 and 232 according to one embodiment can be disposed around the first and second light receivers 221 and 222, respectively, and can measure the illuminance of the red light of the first wavelength range and that of the infrared light of the second wavelength range.

Referring further to FIG. 5, the calculator 250 according to one embodiment can generate a first PPG signal corresponding to the sensed light of the first wavelength range and a second PPG signal corresponding to the sensed light of the second wavelength range. Further, the calculator 250 according to one embodiment can calculate a blood oxygen saturation level of the user with reference to the first and second PPG signals generated as above.

Referring further to FIG. 5, the controller 270 according to one embodiment can adaptively adjust the brightness (or intensity) of the red light of the first wavelength range or the infrared light of the second wavelength range generated by the first or second light emitter 211 or 212, such that the illuminance of the red light of the first wavelength range or the infrared light of the second wavelength range measured by the first or second illuminance sensor 231 or 232 matches a predetermined reference illuminance.

Next, the communicator 260 according to one embodiment can function to enable the PPG signal measurement apparatus 200 to communicate with an external device.

Lastly, the controller 270 according to one embodiment can function to control data flow among the light emitter 210, the light receiver 220, the illuminance sensor 230, the filter 240, the calculator 250, and the communicator 260. The controller 270 can control inbound data flow or data flow among the respective components of the PPG signal measurement apparatus 200, so that the light emitter 210, the light receiver 220, the illuminance sensor 230, the filter 240, the calculator 250, and the communicator 260 can carry out their particular functions, respectively.

The embodiments as described above can be implemented in the form of program instructions that can be executed by various computer components, and can be stored on a non-transitory computer-readable recording medium. The non-transitory computer-readable recording medium can include program instructions, data files, data structures and the like, separately or in combination. The program instructions stored on the non-transitory computer-readable recording medium can be specially designed and configured for the present invention and/or can be known and available to those skilled in the computer software field. Examples of the non-transitory computer-readable recording medium include the following: magnetic media such as hard disks, floppy disks and magnetic tapes; optical media such as compact disk-read only memory (CD-ROM) and digital versatile disks (DVDs); magneto-optical media such as floptical disks; and hardware devices such as read-only memory (ROM), random access memory (RAM) and flash memory, which are specially configured to store and execute program instructions. Examples of the program instructions include not only machine language codes created by a compiler or the like, but also high-level language codes that can be executed by a computer using an interpreter or the like. The above hardware devices can be configured to operate as one or more software modules to perform the processes of the present invention, and vice versa.

Although the present invention has been described in terms of specific items such as detailed elements as well as the limited embodiments and the drawings, they are only provided to help more general understanding of the invention, and the present invention is not limited to the above embodiments. It will be appreciated by those skilled in the art to which the present invention pertains that various modifications and changes can be made from the above description.

Therefore, the scope of the present invention is not limited to the above-described embodiments, and the entire scope of the appended claims and their equivalents will fall within the scope and spirit of the invention.

What is claimed is:

1. A method for measuring photoplethysmography (PPG) signals, comprising:
irradiating light of a first wavelength range and light of a second wavelength range onto a body part of a user, respectively;
sensing light of the first wavelength range entering through a first filter and light of the second wavelength range entering through a second filter, respectively, and measuring a first illuminance of the light of the first wavelength range entering through the first filter and a second illuminance of the light of the second wavelength range entering through the second filter, respectively; and
generating a first PPG signal corresponding to the sensed light of the first wavelength range and a second PPG signal corresponding to the sensed light of the second wavelength range,
wherein, in the irradiating step, brightness of at least one of the light of the first wavelength range and the light of the second wavelength range irradiated onto the body part of the user is adjusted such that a difference between a predetermined reference illuminance and at least one of the first and second measured illuminances is less than a predetermined level.

2. The method of claim 1, wherein, in the irradiating step, the brightness of at least one of the light of the first wavelength range and the light of the second wavelength range irradiated onto the body part of the user is increased when at least one of the first and second measured illuminances is less than the predetermined reference illuminance.

3. The method of claim 1, wherein, in the generating step, an intensity of at least one of the first and second PPG signals is corrected with reference to a relative ratio of the predetermined reference illuminance and at least one of the first and second measured illuminances when at least one of the first and second measured illuminances exceeds the predetermined reference illuminance.

4. The method of claim 1, further comprising:
calculating a blood oxygen saturation level in the body part of the user with reference to the first and second PPG signals.

5. The method of claim 1, wherein the first and second filters selectively transmit the light of the first wavelength range and the light of the second wavelength range, respectively.

6. The method of claim 1, wherein the first wavelength range includes a wavelength range of about 490 nm to 780 nm, and the second wavelength range includes a wavelength range of about 800 nm to 980 nm.

7. The method of claim 1, wherein the light of the first wavelength range and the light of the second wavelength range irradiated onto the body part of the user are irradiated onto the body part of the user through the first and second filters, respectively.

8. A non-transitory computer-readable recording medium having stored thereon a computer program for executing the method of claim 1.

9. An apparatus for measuring photoplethysmography (PPG) signals, comprising:
a first light emitter and a second light emitter configured to irradiate light of a first wavelength range and light of a second wavelength range onto a body part of a user, respectively;
a first light receiver and a second light receiver configured to sense light of the first wavelength range entering through a first filter and light of the second wavelength range entering through a second filter, respectively;
a first illuminance sensor and a second illuminance sensor configured to measure a first illuminance of the light of the first wavelength range entering through the first filter and a second illuminance of the light of the second wavelength range entering through the second filter, respectively;
a calculator configured to generate a first PPG signal corresponding to the sensed light of the first wavelength range and a second PPG signal corresponding to the sensed light of the second wavelength range; and
a controller configured to adjust brightness of at least one of the light of the first wavelength range and the light of the second wavelength range irradiated onto the body part of the user, such that a difference between a predetermined reference illuminance and at least one of the first and second measured illuminances is less than a predetermined level.

10. The apparatus of claim 9, wherein the controller increases the brightness of at least one of the light of the first wavelength range and the light of the second wavelength range irradiated onto the body part of the user when at least one of the first and second measured illuminances is less than the predetermined reference illuminance.

11. The apparatus of claim 9, wherein the calculator is configured to correct an intensity of at least one of the first and second PPG signals with reference to a relative ratio of the predetermined reference illuminance and at least one of the first and second measured illuminances when at least one of the first and second measured illuminances exceeds the predetermined reference illuminance.

12. The apparatus of claim 9, wherein the calculator is configured to calculate a blood oxygen saturation level in the body part of the user with reference to the first and second PPG signals.

13. The apparatus of claim 9, wherein the first and second filter are configured to selectively transmit the light of the first wavelength range and the light of the second wavelength range, respectively.

14. The apparatus of claim 9, wherein the first wavelength range includes a wavelength range of about 490 nm to 780 nm, and the second wavelength range includes a wavelength range of about 800 nm to 980 nm.

15. The apparatus of claim 9, wherein the light of the first wavelength range and the light of the second wavelength range irradiated onto the body part of the user are configured to be irradiated onto the body part of the user through the first and second filters, respectively.
